(19) United States
(12) Patent Application Publication
(10) Pub. No.: US 2007/0160642 A1
(43) Pub. Date: Jul. 12, 2007

(54) Implantable medical devices coated with a polymer-bound superoxide dismutase mimic

(76) Inventor: Stephen Dirk Pacetti, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/332,683

(22) Filed: Jan. 12, 2006

(51) Int. Cl. A61K 31/513; A61K 31/445; A61K 31/421; A61K 31/4015; A61F 2/02

(52) U.S. Cl. 424/423; 514/269; 514/327; 514/376; 514/424

(57) ABSTRACT

This invention relates to implantable medical devices having a coating comprising small molecule superoxide dismutase mimics bonded to biocompatible durable polymers and to uses thereof in the treatment or prevention of vascular diseases.

### FIELD

This invention is directed to the fields of organic chemistry, polymer chemistry, medicinal chemistry, materials science and medical devices.

### BACKGROUND

The discussion that follows is intended solely as background information to assist in the understanding of this invention; nothing in this section is intended to be, nor is it to be construed as, prior art to the invention.

Radical oxygen species (ROS) formed in the body can cause serious damage to physiological systems. For instance, hydroxyl radicals are extremely reactive and can damage cell membranes and lipoproteins, the proteins that carry cholesterol and fat in the blood stream. In fact, proteins in general are susceptible to oxidative damage which can result in loss of enzymatic function or deleterious alteration of that function. DNA is known to be regularly under oxidative attack, it having been estimated that DNA is each cell of the human body experiences 10,000 oxidative “hits” a day leading to the formation of numerous oxidative lesions. While there are numerous repair enzymes that operate to remove these lesions, repair is not always total and some lesions can persist and cause DNA mutations that can lead to, among other diseases, to cancer.

Another ROS is peroxynitrite which is not only a damaging oxidative species in its own right but more importantly results from the oxidation of nitric oxide, an important component of the body's chemistry. Nitric oxide participates in numerous beneficial biochemical pathways such as signaling smooth muscle cells to not proliferate, which in turn inhibits restenosis and contributes to the stabilization of vulnerable plaque.

Most ROS in the body result from reactions involving the superoxide radical. For instance, superoxide reacts with nitric oxide to form peroxynitrite radicals and with water to form hydroxyl radicals. The superoxide radical itself is formed as a normal function of mitochondrial respiratory chains and its biosynthesis is a natural product of normal aerobic metabolism. Thus, even healthy individuals will at any particular time have an abundance of superoxide in their systems. When an individual's system is challenged by foreign objects such as bacteria, air-borne particulates or surgically implanted medical devices, leukocytes are recruited to the invasion site and there generate very large amounts of superoxide as part of the body's defense mechanism against such insults. Inflammation is a common side effect of this leukocyte/superoxide activity.

Undesirable or unneeded superoxide is disposed of by superoxide dismutase (SOD), an enzyme that converts superoxide into less reactive hydrogen peroxide and oxygen. SOD mimics (SOD-m) are small organometallic or organic molecules that catalyze the same conversion. A class of small molecule SOD-m compounds is the aminoxyls, for example 2, 2,6,6-tetramethyl-4-aminopiperidineoxyl, which has been found to possess SOD-m activity.

The present invention provides SOD-m compounds covalently bonded to durable polymers that are coated on implantable medical devices so as to provide long term localized superoxide control.

### SUMMARY

Thus, an aspect of this invention is an implantable medical device comprising:

a biocompatible durable polymer disposed over a surface of the device, wherein:

the biocompatible durable polymer comprises a functional group selected from the group consisting of —OH, —NH₂, —SH, —C(O)OH, —C(O)OR, —NHNH₂, —C(O)NHNH₂, —F, —Cl, —Br, —I, —NCO, —NCS, —CHO, —C(O)CH═CH₂, —SO₂CH═CH₂, aziridyl, oxiranyl and —C(O)OC(O)R, wherein R is 1C-5C alkyl; and,
   a superoxide dismutase mimic covalently bonded to the biocompatible durable polymer, comprising a compound having the chemical structure: 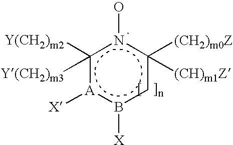
wherein:
n is 0 or 1;
the dashed circle indicates that the ring can be aromatic or non-aromatic;
wherein:
if n is 0 and the ring is aromatic:
   A and B are selected from the group consisting of carbon, oxygen, sulfur and nitrogen provided that if either A or B is oxygen or sulfur, the other is carbon or nitrogen;
   Y′(CH₂)ₘ₃ and Z′(CH₂)ₘ₁ do not exist;
   m₀ and m₂ are independently 0, 1, 2, 3, 4 or 5;
   if A or B is oxygen or sulfur X′ or X does not exist;
   if A or B or both are nitrogen, X′ or X or both either do not exist or one does not exist and the other is hydrogen;
if n is 0 and the ring is not aromatic:
   A and B are selected from the group consisting of carbon, oxygen, nitrogen and sulfur;
   m₀, m₁, m₂, and m₃ are independently 1, 2, 3, 4 or 5;
   if A or B is oxygen or sulfur, X′ or X does not exist;
if n is 1 and the ring is aromatic:
   A and B are independently selected from the group consisting of carbon and nitrogen;
   If A or B is nitrogen, X′ or X does not exist;
   Y′(CH₂)ₘ₃ and Z′(CH₂)ₘ₁ do not exist;
   m₀ and m₂ are independently 0, 1, 2, 3, 4, or 5;
if n is 1 and the ring is not aromatic:
   A and B are independently selected from the group consisting of carbon, nitrogen, oxygen and sulfur;
   m₀, m₁, m₂ and m₃ are independently 1, 2, 3, 4 or 5;
   if A or B is oxygen or sulfur, X′ or X does not exist;
one of X, X′, Y, Y′, Z or Z′ is a complementary functional group selected from the group consisting of —OH, —NH₂, —SH, —C(O)OH, —C(O)OR, —NHNH₂, —C(O)NHNH₂, —Cl, —Br, —NCO, —NCS, —CHO, —C(O)CH═CH₂, —SO₂CH═CH₂, aziridyl, oxiranyl and —C(O)OC(O)R, wherein:
   R is 1C-5C alkyl,
the remaining of X, X′, Y, Y′, Z or Z′ are independently selected from the group consisting of hydrogen, optionally substituted 1C-10C alkyl, optionally substituted 3C-6C alicyclyl, optionally substituted phenyl, optionally substituted heteroaryl and optionally substituted heteroalicyclyl, wherein:
   the optional substituent is selected from the group consisting of —OR′, —NR′R″, —F, —Cl, —Br, —I, and unsubstituted 1C-4C alkyl; or,
   one or Y′(CH₂)ₘ₃ or Z′(CH₂)ₘ₁ may be replaced with a double bond between the carbon to which it was bonded and the aminoxyl nitrogen; or
   Y and X′ taken together can form an unsubstituted aryl, unsubstituted heteroaryl, unsubstituted alicyclyl or unsubstituted heteroalicyclyl fused ring.

In an aspect of this invention, each group other than the one designated as comprising the complementary function group is unsubstituted.

In an aspect of this invention, the superoxide dismutase mimic is selected from the group consisting of:

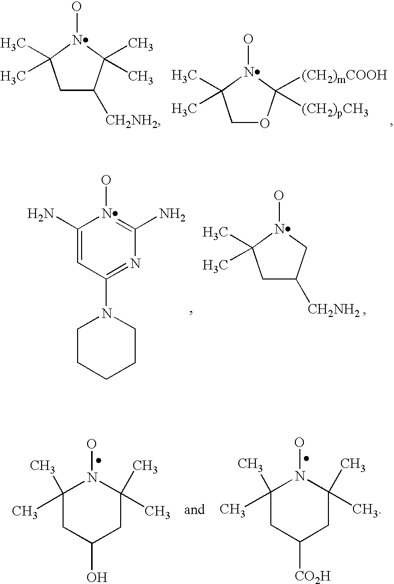

In an aspect of this invention, the biocompatible durable polymer is selected from the group consisting of a polyacrylate, a polymethacryate, a polyurea, a polyesteramide, a polyurethane, a polyolefin, a polyvinylether, a polyvinylaromatic, a polyvinylester, a polyacrylonitrile, an alkyd resin, a polysiloxane, an epoxy resin and a mixture of two or more of the preceding.

In an aspect of this invention, the biocompatible durable polymer is an acrylate or a methacrylate polymer.

In an aspect of this invention, the biocompatible durable polymer is a polyesteramide.

In an aspect of this invention, the biocompatible durable polymer/superoxide dismutase mimic comprises a reservoir layer.

In an aspect of this invention, the biocompatible durable polymer/superoxide dismutase mimic comprises a topcoat layer.

In an aspect of this invention, an implantable medical device wherein the biocompatible durable polymer/superoxide dismutase mimic comprises a topcoat layer, further comprises a reservoir layer.

In an aspect of this invention, the above reservoir layer comprises one or more bioactive agents.

In an aspect of this invention, the above reservoir layer further comprises one or more biobeneficial agents.

In an aspect of this invention, the topcoat layer further comprises one or more biobeneficial agents.

In an aspect of this invention, the implantable medical device is a stent.

In an aspect of this invention, the implantable medical device further comprises a primer layer.

An aspect of this invention is a method of treating or preventing a disease of the vascular system comprising locating the implantable medical device of this invention at the site of the disease.

In an aspect of this invention, the disease is selected from the group consisting of atherosclerosis, restenosis and vulnerable plaque.

### DETAILED DESCRIPTION

In the discussion that follows, it is understood that, with regard to various aspects of this invention, singular implies plural and visa versa. For example, “a bioactive agent” or “the bioactive agent” refers to a single bioactive agent or to a plurality of bioactive agents; “a polymer” or “the polymer”, each refers to a single polymer or a plurality of polymers, etc., unless expressly stated otherwise.

Superoxide dismutase is an enzyme that converts extremely reactive, potentially physiologically harmful superoxide radicals (O₂⁻) to hydrogen peroxide (H₂O₂) and oxygen (O₂). It is believed that superoxide radicals may become overabundant in atherosclerotic lesions following stent implantation. Control of superoxide, then, may help repress inflammation at the implantation site and thereby reduce or prevent the occurrence of restenosis.

As used herein a superoxide dismutase mimic (SOD-m) refers to a non-enzymatic organic aminoxyl compound that performs essentially the same function in vivo as superoxide dismutase. The compound may be aromatic or non-aromatic.

As used herein, an implantable medical device (IMD) refers to any type of appliance that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of IMDs include, without limitation, implantable cardiac pacemakers and defibrillators; leads and electrodes for the preceding; implantable organ stimulators such as nerve, bladder, sphincter and diaphragm stimulators; cochlear implants; prostheses; vascular grafts; self-expandable stents; balloon-expandable stents; stent-grafts; grafts; artificial heart valves and cerebrospinal fluid shunts. The IMD may be intended primarily to perform one of the above tasks or it may be used as an adjunct to other therapeutic modalities with its primary purpose being the delivery of the SOD-m of this invention to a particular site in a patient's vascular system. The IMD may be constructed of any biocompatible material capable of being coated with an adherent layer containing a polymer-SOD-m of this invention.

For example, an IMD useful with this invention may be made of one or more biocompatible metals or alloys including, but not limited to, cobalt-chromium alloy (ELGILOY, L-605), cobalt-nickel alloy (MP-35N), 316L stainless steel, high nitrogen stainless steel, e.g., BIODUR 108, nickel-titanium alloy (NITINOL), tantalum, platinum, platinum-iridium alloy, gold and combinations thereof.

Alternatively, the IMD may be made of a biocompatible, relatively non-biodegradable polymer including, but not limited to, a polyacrylate, a polymethacryate, a polyurea, a polyurethane, a polyolefin, a polyvinylhalide, a polyvinylidenehalide, a polyvinylether, a polyvinylaromatic, a polyvinylester, a polyacrylonitrile, an alkyd resin, a polysiloxane and an epoxy resin.

As used herein, “biocompatible” refers to a polymer that, both in its as-synthesized state and with regard to its biodegradation products, is not, or at least is minimally, toxic to living tissue; does not, or at least minimally and reparably, injure living tissue; and/or does not, or at least minimally and/or controllably, cause an immunological reaction in living tissue.

As used herein, a “durable” polymer refers to a polymer that degrades extremely slowly in a physiological environment, that is, the environment that exists within the body of a patient, including but not limited to physiological pH and temperature, the presence of enzymes and the like. Extremely slowly means that the polymer will exhibit no discernable degradation for at least several months after implantation, for several years after implantation or, alternatively, for the life-time of the recipient of the device. Examples of durable polymers include but are not limited to polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins. Presently preferred durable polymers include polyacrylates, polymethyacrylates and polyesteramides.

As used herein, the “vascular system” refers to the arteries, veins and capillaries that transport blood throughout the body. This includes, without limitation, the cardiovascular system, the carotid artery system and the peripheral vascular system and the veins that complete the circulatory system between each of the foregoing and the heart. The cardiovascular system is the general circulatory system between the heart and all parts of the body. The carotid system supplied blood to the brain. The peripheral vascular system carries blood to and from the peripheral organs such as, without limitation, the arms, legs, kidneys and liver.

As used herein, “vascular disease” refers to a coronary artery disease, a carotid artery disease and/or a peripheral artery disease as such are currently known or as such may become known in the future.

As used herein, a “layer” refers to a thin, preferably at present from about 0.1 to about 100 mm thick, homogeneous, continuous (at least insofar as the surface being coated with the layer is continuous) deposition of a substance onto a surface.

As used herein, a “surface” of an implantable medical device refers to an outer surface that is in direct contact with the external environment, an inner surface if the device has a lumen (the luminal surface) and/or to the edges that connect the outer surface to the luminal surface.

As used herein, to “dispose” a substance over the surface of a device means to form a layer of the substance on the surface of the device or on the surface formed by a previously disposed substance. For example, a primer layer may be applied directly to the surface of a device and then a reservoir layer may be applied to the surface of the primer layer. The layer can be formed by any means presently known or as such may become known in the future including at present, without limitation, spraying, dipping, electro-deposition, roll coating, brushing, direct droplet application and molding.

As used herein, to dispose a substance “over” a surface of a device or over a surface of another layer means that the disposed layer is applied atop the surface of other layer but not necessarily in direct contact with it. That is, there may be one or more additional layers between the disposed layer and the indicated surface such as, without limitation, a spacing layer, a drug release-timing layers, etc.

As used herein, a “primer layer” refers to a layer of a substance, often a polymer, applied directly onto a surface of an implantable medical device to improve the adhesion of a subsequently applied layer. Useful primers include polymers such as, without limitation, polyesteramides (PEAs), polyacrylates and methacrylates (e.g., poly(butyl methacrylate), in particular at present poly(n-butyl methacrylate) and copolymers and combinations thereof.

As use herein, a “reservoir layer” refers to a layer disposed over a surface of an implantable medical device wherein the layer has dispersed within its three-dimensional structure one or more substances selected from a SOD-m of this invention and a bioactive agent. If the topcoat layer does not contain a SOD-m of this invention then the reservoir layer must contain such and, optionally, may contain a bioactive agent as well. If the topcoat layer does contain a SOD-m, then the reservoir layer contains only an optional bioactive agent.

A presently preferred implantable medical device of this invention is a stent. A stent may be self-expandable or balloon expandable. Any type of stent currently known, or as may become known in the future, may be coated with a SOD-m of this invention. A common use of stents is maintenance of patency in a blood vessel that has been surgically restored after having been narrowed or closed due to diseases such as, without limitation, tumors (in, for example, bile ducts, the esophagus, the trachea/bronchi, etc.), benign pancreatic disease, coronary artery disease, carotid artery disease and peripheral arterial disease. Specific diseases include, without limitation, atherosclerosis, re-stenosis and vulnerable plaque.

Restenosis refers to the re-narrowing or blockage of an artery (i.e., the recurrence of a stenosis) at the same site where angioplasty was previously performed. It is usually due to thrombosis accompanied by renewed smooth muscle cell proliferation. Prior to the advent of implantable stents to maintain the patency of vessels opened by angioplasty, restenosis occurred in 40-50% of patients within 3 to 6 months of undergoing the procedure. Post-angioplasty restenosis before stents was due primarily to thrombosis or blood-clotting at the site of the procedure. While the use of IIb-IIIa anti-platelet drugs such as abciximab and epifabatide, which are anti-thrombotic, reduce the occurrence of post-procedure clotting and stents reduce it even further (although stent placement can itself result in thrombosis), stents are also susceptible to restenosis due to abnormal tissue growth at the placement site. This type of restenosis tends to also occur at 3 to 6 months after stent placement but it is not affected by the use of anti-clotting drugs. Thus, alternative therapies are continuously being sought to mitigate, preferably eliminate, this type of restenosis. Drug eluting stents which release a variety of therapeutic agents at the site of stent placement have been in use for some time. Particularly useful have been drug-eluting stents that release sirolimus and more recently everolimus at the site of stent placement. Currently 17-allylamino-17-demethoxygeldanamycin (17-AAG) has found use as a potent inhibitor or such restenosis. It is expected that the SOD-m of this invention will also be useful to treat or prevent restenosis.

A vulnerable plaque refers to an atheromatous plaque that has a very thin wall separating it from the lumen of an artery. The thinness of the wall renders the plaque susceptible or vulnerable to rupture. When the plaque ruptures, tissue debris is released into the arterial lumen and is transported by blood flow to other parts of the vasculature where the size of the debris particles causes them to be trapped at smaller vessels such as capillaries resulting in obstruction with potential serious consequences. Furthermore, rupture of an atheroma may result in bleeding from the lumen of the artery into the tissue of the atheroma resulting in an increase in size of the atheroma to the point that it may narrow or completely obstruct the lumen. In addition, the formation of blood clots at the site of atheroma rupture may itself result in narrowing or complete blockage of the lumen.

Of course, the primary purpose of a stent may simply be the delivery of a SOD-m of this invention to a particular site in the vascular system of a patient. As used herein, to “locate” an implantable medical device at a site of a vascular disease refers to the delivery of the device usually by means of a catheter to or near a site diagnosed to be afflicted with atherosclerosis or engaged in restenosis or to a site diagnosed or suspected to be the location of a vulnerable plaque and to the disengagement of the device from the catheter, leaving it at or near the affected site.

As used herein “bioactive agent” refers to any substance that is of medical or veterinary therapeutic, prophylactic or diagnostic utility. “Amenable to” localized delivery means that the bioactive agent is sufficiently stable to withstand the formulation procedures employed to fabricate an IMD coated with a bioactive agent-releasing layer of this invention, is sufficiently stable to remain intact in the layer until delivery near the site of release and is capable of being released from the coating layer under physiological conditions of temperature, pH, ionic strength, etc. While a SOD-m is, of course, a bioactive agent as defined above, as used herein, “bioactive agent” refers to a substance other than a SOD-m. SOD-m is expressly dealt with separately from other bioactive agents.

As used herein, a “therapeutic agent” refers to a SOD-m or bioactive agent that, when administered to a patient, will cure, or at least relieve to some extent, one or more symptoms of, a disease.

As used herein, a “prophylactic agent” refers to a SOD-m or bioactive agent that, when administered to a patient, either prevents or retards the occurrence of a disease in the first place or, if administered subsequent to a therapeutic agent, prevents or retards the recurrence of the disease.

Bioactive agents that may be used herein include, without limitation:

antiproliferative drugs such as actinomycin D, or derivatives or analogs thereof. Actinomycin D is also known as dactinomycin, actinomycin IV, actinomycin I₁, actinomycin X₁, and actinomycin C₁;

antineoplastics or antimitotics such as, without limitation, paclitaxel, docetaxel, methotrexate, azathioprine, vincristine, vinblastine, fluorouracil, doxorubicin hydrochloride, and mitomycin;

antiplatelet, anticoagulant, antifibrin, and antithrombin drugs such as, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin, and thrombin;

cytostatic or antiproliferative agents such as, without limitation, angiopeptin; angiotensin converting enzyme inhibitors such as captopril, cilazapril or lisinopril; calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide;

antiallergic agent such as, without limitation, permirolast potassium;

other therapeutic agents such as, without limitation, eestradiol, Biolimus™, alpha-interferon, genetically engineered epithelial cells, tacrolimus, clobetasol, dexamethasone and its derivatives, and rapamycin, its derivatives and analogs such as 40-O-(2-hydroxyethyl)rapamycin (EVEROLIMUS®), 40-O-(3-hydroxypropyl)rapamycin, 40-O-[2-(2-hydroxyethoxy)]ethyl-rapamycin, and 40-O-tetrazolylrapamycin and 17-AAG.

If desired, a SOD-m-containing layer of this invention may optionally include a biobeneficial agent in addition to or instead of an optional bioactive agent. A biobeneficial agent is one that beneficially affects an IMD by, for example, reducing the tendency of the device to protein foul, increasing the hemocompatibility of the device, and/or enhancing the non-thrombogenic, non-inflammatory, non-cytotoxic, non-hemolytic, etc. characteristics of the device.

As used herein, “biocompatible durable polymer/superoxide dismutase mimic” refers to the compound obtained after the functional group of the biocompatible durable polymer has reacted with the complementary functional group of the SOD-m to covalently bond the two entities together.

Representative biobeneficial materials include, but are not limited to, polyethers such as poly(ethylene glycol) (PEG) and poly(propylene glycol); copoly(ether-esters) such as poly(ethylene oxide-co-lactic acid); polyalkylene oxides such as poly(ethylene oxide) and poly(propylene oxide); polyphosphazenes, phosphoryl choline, choline, polymers and co-polymers of hydroxyl bearing monomers such as hydroxyethyl methacrylate, hydroxypropyl methacrylate, hydroxypropylmethacrylamide, poly(ethylene glycol) acrylate, 2-methacryloyloxyethylphosphorylcholine (MPC) and n-vinyl pyrrolidone (VP); carboxylic acid bearing monomers such as methacrylic acid, acrylic acid, alkoxymethacrylate, alkoxyacrylate, and 3-trimethylsilylpropyl methacrylate; polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functionalized poly(vinyl pyrrolidone); biomolecules such as fibrin, fibrinogen, cellulose, starch, collagen, dextran, dextrin, hyaluronic acid, heparin, glycosamino glycan, polysaccharides, elastin, chitosan, alginate, silicones, PolyActive™, and combinations thereof. PolyActive™ refers to a block copolymer of poly(ethylene glycol) and poly(butylene terephthalate).

The amount of SOD-m and/or bioactive agent in a layer will depend on the required MEC (minimum effective concentration) of the SOD-m and/or bioactive agent and the length of time over which it is desired that a concentration of the SOD-m and/or bioactive agent that is equal to or greater than the MEC is to be maintained at the site. For many bioactive agents the MEC will be described in the literature accompanying the commercial drug. For experimental bioactive agents, or those for which the MEC by localized delivery is not known and for the SOD-m of this invention, the MEC can be empirically determined using techniques well-known to those skilled in the art.

As used herein, a “patient” refers to any organism that can benefit from the use of a bioactive agent releasing IMD. In particular at present, patient refers to a mammal such as, without limitation, a cat, dog, horse, cow, pig, sheep, rabbit, goat or, most preferably at present, a human being.

The IMD may further comprise a topcoat layer in addition to the reservoir layer. As used herein, a topcoat layer refers to a thin layer of polymeric material that is disposed over the reservoir layer. The topcoat layer may be in direct contact with the environment. Occasionally, however, a finishing layer may be applied to the surface of a topcoat but such a layer is usually extremely thin and has little effect other than to present a smoother, possibly more lubricious surface to the environment. As used herein, a thin layer refers to a layer that has a thickness of from about 0.1 to about 20 microns. The topcoat may be present for the purpose of protecting the layer or layers beneath it from the environment until the IMD is in place at the target location. The topcoat layer may also, or in the alternative, assist in controlling the rate of release of an optionally included bioactive agent. A topcoat layer can have dispersed within it the same or a different bioactive agent from that in the reservoir layer that is to be released rapidly at the target site upon implantation (burst release). The topcoat layer may also include an optional biobeneficial agent to assist in the biocompatibility of the IMD and, of course, if present, the topcoat layer can include a SOD-m of this invention.

As used herein, “alkyl” refers to a straight or branched chain fully saturated (no double or triple bonds) hydrocarbon group. An alkyl group of this invention comprises from 1 to 20 carbon atoms. Preferably at present an alkyl group herein comprise 1 to 10 carbon atoms and more preferably at present, from 1 to 5 carbon atoms. Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

As used herein, “mC to nC” in which “m” and “n” are integers refers to the number of carbon atoms in an alkyl group. Thus, for example, a “(1C to 4C alkyl” refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)—, and (CH₃)₃CH—. If no “m” and “n” are designated, that is the term “alkyl” is used alone, the broadest range described in these definitions applies.

As used herein, “aromatic” refers to a five- or six-membered ring which has a fully delocalized π-electron system. An aromatic six-member ring can have only carbon atoms in the ring in which case it would be an “aryl” ring or it may have carbon and nitrogen atoms in the ring, in which case it would be “heteroaryl.” An aromatic five-member ring must have at least one heteroatom (oxygen, sulphur or nitrogen) in the ring, the remaining four atoms being carbon or any combination of carbon and nitrogen. “Phenyl” refers to a 6-membered all carbon aromatic ring. The ring may be optionally substituted with one or two groups independently selected from the group consisting of unsubstituted alkyl, F, Cl, Br, I, —CN, —NO₂, —OR and —NR₂ wherein R is hydrogen or unsubstituted alkyl.

As used herein, “non-aromatic” refers to a ring system that may or may not contain double bonds but if it does contain double bonds there are not present in sufficient number to form a fully delocalized π-electron system.

As used herein, “alicyclyl” refers to an all-carbon atom non-aromatic ring. Examples of alicyclic ring systems include, without limitation, cyclopropane, cyclobutane, cyclopentane, cyclopentene, cyclopentadiene, cyclohexane, cyclohexene, cyclohexadiene, cycloheptane and cyclooctane. The alicyclyl ring may optionally be substituted with one or two groups independently selected from the group consisting of unsubstituted alkyl, F, Cl, Br, I, —CN, —NO₂, —OR and —NR₂ wherein R is hydrogen or unsubstituted alkyl.

As used herein, “heteroalicyclyl” refers to a non-aromatic ring that may contain carbon, nitrogen, oxygen and/or sulphur. Examples of heteroalicylcyl rings include, without limitation, aziridine, oxirane, tetrahydrofuran, tetrahydrothiophene, pyrrolidine, piperidine, morpholine, dioxane and piperazine. The carbon atoms of the heteroalicyclyl group may optionally be substituted with a group selected from the group consisting of unsubstituted alkyl, F, Cl, Br, I, —CN, —NO₂, —OR and —NR₂ wherein R is hydrogen or unsubstituted alkyl. A nitrogen of a heteroalicyclyl group may optionally be substituted with an unsubstituted alkyl. Oxygen and sulphur of heteroalicyclic groups cannot be further substituted.

As used herein, when it is stated that two adjacent groups on a ring are “taken together,” it means that a covalent bond is formed between the groups so as to form a second ring which is then said to be “fused” to the first ring. “Fused” is means that two rings share an adjacent pair of atoms independently selected from carbon and nitrogen; that is, the carbon and/or nitrogen atoms participate in both rings. The new ring may be aromatic or non-aromatic.

By “optionally substituted” is meant that a group so designated may be composed of only the atoms of the base structure, that is, carbon and hydrogen in alkyls and carbon, nitrogen, oxygen and sulphur in aromatic and non-aromatic rings with hydrogens appended therefrom (unsubstituted) or one of more of the hydrogen atoms may be replaced with a different atom that is or forms a part of a substituent group.

As used herein, “aminoxyl” refers to the chemical structure RR′NO wherein R and R′ are aliphatic moieties or, taken together, form with the nitrogen, a aromatic or non-aromatic ring.

As used herein, a “functional group” is a reactive chemical moiety that is capable of reacting with another reactive moiety, termed herein a “complementary functional group,” to form a third moiety, termed herein the “product group” that covalently bonds the molecule to which the functional group was initially attached with the molecule to which the complementary functional group was attached. Examples of functional groups and complementary functional groups and, in [square brackets], the product group resulting from their reaction, include without limitation, ROH and R′C(O)OH[C(O)OR]; RNH₂ and R′C(O)OH[R′CONHR]; ROH and R′C(O)OC(O)R″[R′—C(O)OR]; RNH₂ and R′C(O)OC(O)R″[R′C(O)NHR]; ROH and R′X[R′OR]; RNH₂ and R′X [R′NHR]; RSH and R′NCO[R′NHC(O)SR]; RNH₂ and R′NCS[R′NHC(S)NHR]; ROH and R′CHO[R′C(OR₂)]; RNH₂ and R′CHO[R′C═NR]; RC(O)OH and R′C(O)OH[R′C(O)OC(O)R]; —RNH₂ and R′C(O)X[R″C(O)NHR]; RSH and R′X [R′SR], RC(O)CH═CH₂ and R′OH[RC(O)CH₂CH₂OR′]; RC(O)CH═CH₂ and R′NH₂[RC(O)CH₂CH₂NHR′]; RC(O)CH═CH₂ and R′SH[RC(O)CH₂CH₂SR′]; RSO₂CH═CH₂ and R′OH[RSO₂CH₂CH₂OR′]; RSO₂CH═CH₂ and R′NH₂[SO₂CH₂CH₂NHR′]; RSO₂CH═CH₂ and R′SH[RSO₂CH₂CH₂SR′]; wherein X is chlorine, bromine or iodine and one of R and R′ is the biocompatible durable polymer and one is the compound.

The compounds of this invention can be synthesized by methods well-known to those skilled in the art. For example, without limitation, if a polyacrylate ester is selected as the biocompatible durable polymer, the moiety used to esterify the acrylic acid monomer could contain the functional group that will be bonded to the SOD-m. With regard to the SOD-m compound, the aminoxyl moiety can be prepared by oxidation of a secondary (AA′NH) or tertiary (AA′A″N) nitrogen with, for example but without limitation, hydrogen peroxide. The synthesis of compounds having any of the other functional groups disclosed herein will be equally apparent to those skilled in the art. In fact, those skilled in the art will immediately recognize other functional groups and complementary functional groups that will react to form product groups that covalently bond a biocompatible durable polymer to a compound. All such functional groups and complementary functional groups are within the scope of this invention.

What is claimed:

1. An implantable medical device comprising:
a biocompatible durable polymer disposed over a surface of the device, wherein:
the biocompatible durable polymer comprises a functional group selected from the group consisting of —OH, —NH₂, —SH, —C(O)OH, —C(O)OR, —NHNH₂, —C(O)NHNH₂, —F, —Cl, —Br, —I, —NCO, —NCS, —CHO, —C(O)CH═CH₂, —SO₂CH═CH₂, aziridyl, oxiranyl and —C(O)OC(O)R, wherein R is 1C-5C alkyl; and,
a superoxide dismutase mimic bonded to the biocompatible durable polymer wherein the superoxide dismutase mimic is a compound having the chemical structure: 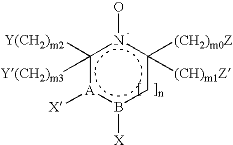
wherein:
n is 0 or 1;
the dashed circle indicates that the ring can be aromatic or non-aromatic;
wherein:
if n is 0 and the ring is aromatic:
A and B are selected from the group consisting of carbon, oxygen, sulfur and nitrogen provided that if either A or B is oxygen or sulfur, the other is carbon or nitrogen;
Y′(CH₂)ₘ₃ and Z′(CH₂)ₘ₁ do not exist;
m₀ and m₂ are independently 0, 1, 2, 3, 4 or 5
if A or B is oxygen or sulfur X′ or X does not exist;
if A or B or both are nitrogen, X′ or X or both either do not exist or one does not exist and the other is hydrogen;
if n is 0 and the ring is not aromatic:
A and B are selected from the group consisting of carbon, oxygen, nitrogen and sulfur;
m₀, m₁, m₂, and m₃ are independently 1, 2, 3, 4 or 5;
if either A or B or both are oxygen or sulfur, X′ or X or both do not exist;
if n is 1 and the ring is aromatic:
A and B are independently selected from the group consisting of carbon and nitrogen;
If A or B is nitrogen, X′ or X does not exist;
Y′(CH₂)ₘ₃ and Z′(CH₂)ₘ₁ do not exist;
m₀ and m₂ are independently 0, 1, 2, 3, 4, or 5;
if n is 1 and the ring is not aromatic:
A and B are independently selected from the group consisting of carbon, nitrogen, oxygen and sulfur;
m₀, m₁, m₂ and m₃ are independently 1, 2, 3, 4 or 5;
if A and/or B is oxygen or sulfur, X′; and/or X does not exist;
one of X, X′, Y, Y′, Z or Z′ is a complementary functional group selected from the group consisting of —OH, —NH₂, —SH, —C(O)OH, —C(O)OR, —NHNH₂, —C(O)NHNH₂, Cl, Br, —NCO, —NCS, —CHO, —C(O)CH═CH₂, —SO₂CH═CH₂, aziridyl, oxiranyl and —C(O)OC(O)R, wherein R is 1C-5C alkyl,
the remaining of X, X′, Y, Y′, Z or Z′ are independently selected from the group consisting of hydrogen, optionally substituted 1C-1C alkyl, optionally substituted 3C-6C alicyclyl, optionally substituted phenyl, optionally substituted heteroaryl and optionally substituted heteroalicyclyl, wherein:
the optional substituent is selected from the group consisting of —OR′, —NR′R″, —F, —Cl, —Br, —I, and unsubstituted 1C-4C alkyl; or,
one of Y′(CH₂)ₘ₃ or Z′(CH₂)ₘ₁ may be replaced with a double bond between the carbon to which it was bonded and the aminoxyl nitrogen; or,
Y and X′, taken together, can form an unsubstituted aryl, unsubstituted heteroaryl, unsubstituted alicyclyl or unsubstituted heteroalicyclyl fused ring.

2. The compound of claim 1, wherein each group other than the one designated as comprising the complementary function group is unsubstituted.

3. The implantable medical device of claim 1, wherein the superoxide dismutase mimic is selected from the group consisting of:

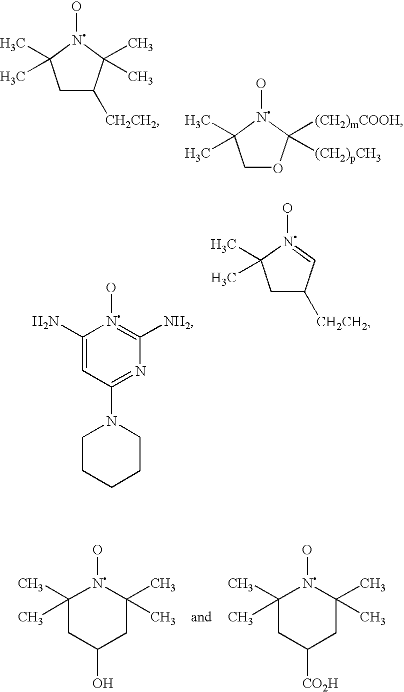

4. The implantable medical device of claim 1, wherein the biocompatible durable polymer is selected from the group consisting of a polyacrylate, a polymethacryate, a polyurea, a polyesteramide, a polyurethane, a polyolefin, a polyvinylether, a polyvinylaromatic, a polyvinylester, a polyacrylonitrile, an alkyd resin, a polysiloxane, an epoxy resin and a mixture of two or more of the preceding.

5. The implantable medical device of claim 4, wherein the biocompatible durable polymer is an acrylate or a methacrylate polymer.

6. The implantable medical device of claim 4, wherein the biocompatible durable polymer is a polyesteramide.

7. The implantable medical device of claim 1, wherein the biocompatible durable polymer with the superoxide dismutase mimic bonded to it comprises a reservoir layer.

8. The implantable medical device of claim 1, wherein the biocompatible durable polymer/superoxide dismutase mimic comprises a topcoat layer.

9. The implantable medical device of claim 8 further comprising a reservoir layer.

10. The implantable medical device of claim 7, wherein the reservoir layer further comprises one or more bioactive agents.

11. The implantable medical device of claim 10, wherein the reservoir layer further comprises one or more biobeneficial agents.

12. The implantable medical device of claim 8, wherein the topcoat layer further comprises one or more biobeneficial agents.

13. The implantable medical device of claim 1, wherein the device is a stent.

14. The implantable medical device of claim 1, further comprising a primer layer.

15. A method of treating or preventing a disease of the vascular system comprising locating the implantable medical device of claim 1 at the site of the disease.

16. The method of claim 15, wherein the disease is selected from the group consisting of atherosclerosis, restenosis and vulnerable plaque.
